(19) United States
(12) Patent Application Publication
(10) Pub. No.: US 2020/0281847 A1
(43) Pub. Date: Sep. 10, 2020

(54) WATER-SOLUBLE POLYMER ADHESIVE LAYERS

(71) Applicant: LTS LOHMANN THERAPIE-SYSTEME AG, Andernach (DE)

(72) Inventors: Michael LINN, Waldbockelheim (DE); Markus MULLER, Troisdorf (DE); Marius BAUER, Andernach (DE)

(21) Appl. No.: 16/765,775

(22) PCT Filed: Nov. 21, 2018
(86) PCT No.: PCT/EP2018/082091

§ 371 Date: May 20, 2020

(30) Foreign Application Priority Data
Nov. 21, 2017 (DE) 10 2017 127 452.9

(51) Int. Cl. A61K 9/00; A61K 9/70; A61K 31/122; A61K 47/32

(52) U.S. Cl. CPC A61K 9/006; A61K 47/32; A61K 31/122; A61K 9/7007

(57) ABSTRACT

Disclosed is a multi-layer oral active-substance film comprising at least two non-adhesive layers each containing at least one hydrophilic polymer, and at least one water-soluble adhesive layer containing at least one water-soluble polymer and at least one plasticiser, wherein at least one of the at least two non-adhesive layers and/or the at least one water-soluble adhesive layer contains a pharmaceutically active substance, and wherein the at least two non-adhesive layers and the at least one water-soluble adhesive layer alternate in such a way that the at least one water-soluble adhesive layer is covered on both sides by at least one non-adhesive layer.

The present invention relates to a multi-layer oral active-substance film, a method for production thereof, and use thereof as a medicinal product.

Oral active-substance films are thin films that contain a pharmaceutically active substance and are placed directly in the oral cavity or against the oral mucosa and dissolve there.

These thin oral active-substance films can be constructed as single- or multi-layer systems. The active substance may be dissolved, emulsified or dispersed in the film.

Multi-layer oral active-substance films are known from the prior art.

Document WO 2011/134846 A1 discloses multi-layer oral active-substance films comprising an active substance-containing layer and a layer containing a substance that is incompatible with the active substance in the active substance-containing layer, these two layers being separated by a further, protective layer situated between these two layers.

Document US 2013/0017235 A1 discloses multi-layer oral active-substance films in which an active substance-containing layer is surrounded by two water-swellable polymer layers.

The multi-layer oral active-substance films known from the prior art are generally produced by a method in which a first layer is produced initially, and, once this has been dried, a second layer is applied to the first layer. Once the second layer has been dried on the first layer, a third layer may then be applied optionally. By means of such a method, multi-layer active-substance films can indeed be provided, but only by the lamination of further layers onto an already existing layer. This has the disadvantage that the pharmaceutically active substance is thermally and chemically loaded to a greater extent on account of the repeated coating. In addition, a bond created by the application of a layer to an already existing layer is often unstable and may easily degenerate. Furthermore, any defects in the coating, in particular defective area densities, propagate through the subsequent coatings.

The aim of the present invention lies in overcoming the above-mentioned disadvantages of the prior art. In particular, the object of the present invention is to provide a multi-layer oral active-substance film in which a plurality of layers (these may be layers of the same or different compositions) are adhered such that any defects in the production of one layer do not influence the other layers, wherein at least one of these layers contains at least one pharmaceutically active substance, such that a firm bond is created between these layers, and the at least one pharmaceutically active substance is not thermally and/or chemically loaded repeatedly. The multi-layered oral active-substance film should preferably dissolve when placed in the oral cavity.

The above aim is addressed by a multi-layer oral active-substance film according to claim 1, which comprises at least two non-adhesive layers each containing at least one hydrophilic polymer, and at least one water-soluble adhesive layer containing at least one water-soluble polymer and at least one plasticiser, wherein at least one of the at least two non-adhesive layers and/or the at least one water-soluble adhesive layer contains a pharmaceutically active substance, and wherein the at least two non-adhesive layers and the at least one water-soluble adhesive layer alternate in such a way that the at least one water-soluble adhesive layer is covered on both sides by at least one non-adhesive layer.

In particular, the inventors have discovered that a water-soluble adhesive layer containing at least one water-soluble polymer and at least one plasticiser, as intermediate water-soluble adhesive layer, can firmly adhere two further layers to one another, which are not sticky per se, and therefore it is possible to achieve the structure of multi-layer oral active-substance films without multiple coatings one above the other.

In addition, the multi-layer oral active-substance films according to the invention have the advantage that each layer is producible separately, so that it also can be stored if necessary, and therefore a multitude of oral active-substance films thus can be produced easily from different combinations of layers.

Furthermore, one advantage lies in the fact that the production of active-substance films having high areal densities, which therefore have a long drying time, can be avoided. For example, an active-substance film with an areal density of 150 g/m² can be replaced by a multi-layer oral active-substance film in which two layers each with an areal density of 75 g/m² are adhered to one another by an intermediate adhesive layer. This is advantageous since the drying time of a layer with an areal density of 75 g/m² is much shorter.

In this context, an adhesive layer is understood to mean a layer that can act as adhesive, as defined in DIN EN 923:2016-03. A non-adhesive layer therefore cannot act as adhesive as defined hereinbefore.

Water-soluble polymers comprise chemically very different, natural or synthetic polymers, of which the common feature is their solubility in water or aqueous media. A precondition for this is that these polymers have a number of hydrophilic groups sufficient for water solubility and are not cross-linked. The hydrophilic groups may be non-ionic, anionic, cationic and/or zwitterionic.

A hydrophilic polymer is a polymer that contains polar or charged groups. These groups may be non-ionic, anionic, cationic or zwitterionic. Hydrophilic polymers are generally water-soluble, but may also be poorly soluble or insoluble in water.

Plasticisers are liquid or solid, indifferent organic substances, preferably with low vapour pressure, which, without chemical reaction, preferably due to their dissolving and swelling capacity, but in some circumstances also without such a capacity, physically interact with highly polymeric substances and can form a homogeneous system therewith. Plasticisers impart certain sought physical properties onto the structures or coatings produced using them, for example lower freezing point, increased deformation capability, increased elastic properties, reduced hardness and possibly increased adhesion. They belong to the plastic additives.

In the multi-layer oral active-substance film according to the invention, the non-adhesive layers and the water-soluble adhesive layer or the water-soluble adhesive layers alternate in such a way that a water-soluble adhesive layer is always present between two non-adhesive layers. In this case the non-adhesive layers may form the outermost layers, so that the multi-layer oral active-substance film according to the invention is not sticky on its outer sides. In principle, oral active-substance films with any number of layers can be provided by means of this structure.

The multi-layer oral active-substance film according to the invention, however, preferably comprises a total of three layers: two non-adhesive layers with an intermediate water-soluble adhesive layer.

Embodiments of the multi-layer oral active-substance film according to the invention in which one of the outermost non-adhesive layers is replaced by a plastic film, such as a film formed from polyethylene terephthalate, polyethylene, polybutylene, polyurethane, polyester, water-insoluble cellulose, such as ethyl cellulose etc., are also conceivable.

In principle, the pharmaceutically active substance may be contained in the multi-layer oral active-substance film according to the invention in each of the layers of the multi-layer oral active-substance film according to the invention. In this case the compatibility of the particular active substance with the material of which a particular layer comprises must be taken into consideration. However, the compatibility of the active substance with the material of which the other layers comprise must also be taken into consideration, since a migration of the active substance in the multi-layer system cannot be ruled out.

The multi-layer oral active substance according to the invention is not limited to the fact that only one pharmaceutically active substance is contained. Multi-layer oral active-substance films in which different pharmaceutically active substances are contained in different layers are conceivable. An individual layer may also contain more than one pharmaceutically active substance.

The present invention is especially advantageous in respect of a multi-layer oral active-substance film which contains different active substances in different layers. In accordance with the invention, these different layers may all be produced separately and then adhered to one another in any combinations by the intermediate adhesive layers in accordance with a modular design. Since the at least one water-soluble adhesive layer, i.e. also a plurality of water-soluble adhesive layers, may contain pharmaceutically active substances, the number of possible combinations can be further increased. Complex multi-layer oral active-substance films are thus also easily accessible.

A multi-layer oral active-substance film that contains at least one pharmaceutically active substance in at least one of the at least two non-adhesive layers is preferred.

In addition, a multi-layer oral active-substance film that contains at least one pharmaceutically active substance in the at least one water-soluble adhesive layer is additionally preferred.

The at least one pharmaceutically active substance, however, may also be provided in any other layer or any other layers and in any combinations of layers.

Generally, each pharmaceutically active substance that is suitable for transmucosal or oral administration may be contained in the multi-layer oral active-substance film according to the invention.

The at least one pharmaceutically active substance is preferably idebenone.

The multi-layer oral active-substance film according to the invention is characterised in that the at least one hydrophilic polymer in the at least two non-adhesive layers comprises polyvinyl alcohol, polyethylene oxide, polyvinylpyrrolidone or copolymers thereof or a polyvinyl caprolactam/polyvinyl acetate/polyethylene glycol copolymer (Soluplus) or a vinylpyrrolidone/vinyl acetate copolymer (Kollidon VA 64) or a cellulose derivative, such as hydroxypropyl cellulose or hydroxypropyl methylcellulose, starch or a starch derivative, shellac, alginic acid, galactomannan, carrageenan and other natural gums, pullulan, xanthan, pectin and other glucans, dextran, poly(meth)acrylates, polyalkylene glycols, carboxyvinyl polymers and/or copolymers thereof.

In an especially preferred embodiment, the at least one hydrophilic polymer comprises polyvinyl alcohol, polyvinylpyrrolidone and/or copolymers thereof, shellac or a polyvinyl caprolactam/polyvinyl acetate/polyethylene glycol copolymer or a vinylpyrrolidone/vinyl acetate copolymer or a cellulose derivative, such as hydroxypropyl cellulose.

In an especially preferred embodiment, the oral thin film according to the invention is preferably characterised in that the at least two non-adhesive layers each comprise a different hydrophilic polymer selected from the group consisting of polyvinyl alcohol, polyethylene oxide, polyvinylpyrrolidone or copolymers thereof, polyvinyl caprolactam/polyvinyl acetate/polyethylene glycol copolymer, vinylpyrrolidone/vinyl acetate copolymer, a cellulose derivative, such as hydroxypropyl cellulose or hydroxypropyl methylcellulose, starch or a starch derivative, shellac, alginic acid, galactomannan, carrageenan and other natural gums, pullulan, xanthan, pectin and other glucans, dextran, poly(meth)acrylates, polyalkylene glycols, carboxyvinyl polymers and/or copolymers thereof.

These hydrophilic polymers have the advantage that they are compatible with a large number of pharmaceutically active substances and in addition are largely safe for a patient whose treatment involves the multi-layer oral active-substance film according to the invention.

These hydrophilic polymers additionally have the advantage that, when dried, they form a thin, stable film which dissolves when applied to the mucosa or placed in the oral cavity and thus releases the active substance. This has the advantage of a rapid availability of the active substance and a residue-free administration of the active substance.

The at least one hydrophilic polymer in the at least two non-adhesive layers may be the same hydrophilic polymer in each case, or a different hydrophilic polymer may be present in each layer.

The multi-layer oral active-substance film according to the invention is additionally preferably characterised in that the at least one water-soluble polymer in the at least one water-soluble adhesive layer comprises shellac, a vinylpyrrolidone/vinyl acetate copolymer, a polyvinyl caprolactam/polyvinyl acetate/polyethylene glycol copolymer, hydroxypropyl cellulose or hydroxypropyl methylcellulose and/or polyvinylpyrrolidone.

The multi-layer oral active-substance film is preferably characterised in that the at least one plasticiser in the at least one water-soluble adhesive layer comprises glycerol, polyethylene glycol, in particular polyethylene glycol 200, sorbitol and/or tributyl citrate.

The at least one plasticiser in the at least one water-soluble adhesive layer especially preferably comprises glycerol, polyethylene glycol 200 and/or tributyl citrate.

Due to the use, preferably in the form of a mixture, of at least one water-soluble polymer and at least one plasticiser, a sticky mixture is created, which, preferably after drying, can be used as adhesive layer in the multi-layer oral active-substance film according to the invention.

The weight ratio of the at least one water-soluble polymer to the at least one plasticiser in the at least one water-soluble adhesive layer is preferably approximately 85 to 50 to approximately 15 to 50, preferably 85 to 65 to approximately 15 to 35, even more preferably approximately 80 to 60 to approximately 20 to 40, even more preferably approximately 80 to 50 to approximately 20 to 50, even more preferably approximately 82 to 68 to approximately 18 to 32 and most preferably approximately 80 to 70 to approximately 20 to 30.

The weight ratio of the at least one water-soluble polymer to the at least one plasticiser in the at least one water-soluble adhesive layer is especially preferably approximately.

If too little or too much plasticiser is used, the mixture thus is either not sticky, or it is not possible to provide a workable material mixture at all.

The at least one pharmaceutically active substance is contained in principle at least in a pharmaceutically effective amount in at least one of the layers of the multi-layer oral active-substance film according to the invention.

The at least one pharmaceutically active substance is preferably provided in at least one of the at least two non-adhesive layers and/or in the at least one water-soluble adhesive layer. It is especially preferred if the pharmaceutically active substance is present in each case in an amount of up to 75 wt. %, preferably in an amount of from 0.01 to 75 wt. %, in relation to the total weight of the layer in question.

The multi-layer oral active-substance film according to the invention is also characterised in that the at least two non-adhesive layers each have an areal density of from 20 to 200 g/m². If the areal density is higher, this has the disadvantage of especially long drying times, which is economically disadvantageous. A film with a lower areal density can only be processed with difficulty, and therefore is not preferred.

Conventional additives such as permeation enhancers, antioxidants, flavourings, taste-masking substances, preservatives, colorants, inert fillers, etc., may additionally be contained in the various layers of the multi-layer oral active-substance film according to the invention.

The present invention also relates to a method for producing a multi-layer oral active-substance film, as defined above, comprising the following steps

(a) providing a first non-adhesive layer containing at least one hydrophilic polymer,

(b) providing a further non-adhesive layer containing at least one hydrophilic polymer,

(c) providing a water-soluble adhesive layer containing at least one water-soluble polymer and at least one plasticiser,

(d) applying the adhesive layer from step (c) to one of the non-adhesive layers from steps (a) or (b) in order to obtain a laminate, and

(e) applying the further non-adhesive layer from steps (a) or (b) to the water-soluble adhesive layer of the laminate from step (d) so that the water-soluble adhesive layer is covered from both sides by at least one non-adhesive layer, at least one of the layers produced in steps (a), (b) and (c) additionally containing at least one pharmaceutically active substance.

The layers a) and b) may be layers of different composition. The layers a) and b), however, may also be layers of the same composition. The layers a) and b) may also be layers that have been obtained from the same starting layer and then have been adhered together in accordance with the above method.

If the multi-layer oral active-substance film comprises a total of more than three layers, the alternate application of adhesive layers and non-adhesive layers is thus continued until the desired number of layers is achieved, with two non-adhesive layers in each case forming the outermost layers of the multi-layer oral active-substance film.

The present invention additionally relates to a multi-layer oral active-substance film obtainable by the method described above.

The present invention also relates to a multi-layer oral active-substance film as described above or obtainable by the above-described method for use as a medicinal product.

The invention will be explained hereinafter on the basis of non-limiting examples.

### EXAMPLE 1

The mixtures of a water-soluble polymer and a plasticiser specified in Table 1 were tested in respect of their stickiness haptically by being pressed on with a finger.

TABLE 1
Example formulations

| | % | | % | |
|---|---|---|---|---|
| Polymer | Polymer | Plasticiser | Plasticiser | Assessment |
| Soluplus | 80 | PEG 200 | 20 | Sticky |
| Soluplus | 70 | PEG 200 | 30 | Sticky |
| Soluplus | 80 | Glycerol | 20 | Sticky |
| Soluplus | 90 | PEG 200 | 10 | Not sticky |
| Soluplus | 90 | Glycerol | 10 | Not sticky |
| Soluplus | 90 | Tributyl | 10 | Not sticky |
| | | citrate | | |
| PVP 30 | 80 | PEG 200 | 20 | Sticky |
| PVP 30 | 70 | PEG 200 | 30 | Sticky |
| PVP 30 | 80 | Glycerol | 20 | Sticky |
| PVP 30 | 90 | PEG 200 | 10 | Not sticky |
| PVP 30 | 90 | Glycerol | 10 | Not sticky |
| PVP 30 | 90 | Tributyl | 10 | Not sticky |
| | | citrate | | |
| Kollidon | 70 | Glycerol | 30 | Sticky |
| VA 64 | | | | |
| Kollidon | 70 | PEG 200 | 30 | Sticky |
| VA 64 | | | | |
| Kollidon | 70 | Tributyl | 30 | Sticky |
| VA 64 | | citrate | | |
| Kollidon | 80 | PEG 200 | 20 | Sticky |
| VA 64 | | | | |
| Kollidon | 80 | Glycerol | 20 | Sticky |
| VA 64 | | | | |
| Kollidon | 40 | PEG 200 | 60 | Material mixture |
| VA 64 | | | | not not possible |
| Kollidon | 40 | Glycerol | 60 | Material mixture |
| VA 64 | | | | not possible |
| Kollidon | 40 | Tributyl | 60 | Material mixture |
| VA 64 | | citrate | | not possible |
| Kollidon | 85 | Glycerol | 15 | Not sticky |
| VA 64 | | | | |
| Kollidon | 85 | PEG 200 | 15 | Not sticky |
| VA 64 | | | | |
| Kollidon | 85 | Tributyl | 15 | Not sticky |
| VA 64 | | citrate | | |
| Shellac | 70 | Glycerol | 30 | Sticky |
| Shellac | 70 | PEG 200 | 30 | Sticky |
| Shellac | 70 | Tributyl | 30 | Sticky |
| | | citrate | | |
| Shellac | 50 | Glycerol | 50 | Sticky |
| HPC | 70 | Glycerol | 30 | Sticky |
| HPC | 70 | PEG 200 | 30 | Sticky |
| HPC | 50 | PEG 200 | 50 | Sticky |

Soluplus: Polyvinyl caprolactam/polyvinyl acetate/polyethylene glycol copolymer (BASF, Germany)
PVP 30: polyvinylpyrrolidone
Kollidon VA 64: vinylpyrrolidone/vinyl acetate copolymer
HPC: hydroxypropyl cellulose
PEG 200: polyethylene glycol 200

### EXAMPLE 2

The production of an active substance-containing layer, consisting of 25% idebenone (micronised) and 75% polyvinyl alcohol (PVA 4-88) with an areal density of 150 g/m², in the case of a single-layer active-substance film, would result in drying times that are not commercially viable due to the use of water as solvent (caused by PVA) and a drying temperature of 45° C. (caused by the low melting point of idebenone).

This problem can be addressed by a multi-layer oral active-substance film according to the invention. The active substance-containing layer is produced with two layers each of 75 g/m², or with one layer that is divided for use as a top side and bottom side, whereby the drying times can be reduced to an acceptable level.

A water-soluble adhesive layer formed of 80% Kollidon VA 64 and 20% PEG 200 is produced separately. This layer is applied to the active substance-containing layer. This adhesive layer is then covered by a further active substance-containing layer.

This results in an overall laminate which contains the desired 150 g/m² active substance-containing layer (as 2×75 g/m²). The adhesive layer in this case has an areal density of 60 g/m².

TABLE 2
Composition of a multi-layer active-substance
film according to the invention.

| | Material | Function | wt. % | Final wt. % |
|---|---|---|---|---|
| Active | PVA 4-88 | Hydrophilic | 75 | 53.57 |
| substance- | | polymer | | |
| containing | | | | |
| non-adhesive | | | | |
| layer | | | | |
| (2 × 75 g/m²) | | | | |
| | Idebenone | Active | 25 | 17.86 |
| | | substance | | |
| Adhesive layer | Kollidon | Water-soluble | 80 | 22.86 |
| (60 g/m²) | VA 64 | polymer | | |
| | PEG 200 | Plasticiser | 20 | 5.71 |

PVA 4-88: polyvinyl alcohol
Kollidon VA 64: vinylpyrrolidone/vinyl acetate copolymer
PEG 200: polyethylene glycol 200

1. A multi-layer oral active-substance film comprising at least two non-adhesive layers each containing at least one hydrophilic polymer, and at least one water-soluble adhesive layer containing at least one water-soluble polymer and at least one plasticiser, wherein at least one of the at least two non-adhesive layers and/or the at least one water-soluble adhesive layer contains a pharmaceutically active substance, characterised in that the at least two non-adhesive layers and the at least one water-soluble adhesive layer alternate in such a way that the at least one water-soluble adhesive layer is covered on both sides by at least one non-adhesive layer.

2. The multi-layer oral active-substance film according to claim 1, characterised in that the at least one pharmaceutically active substance is contained in at least one of the at least two non-adhesive layers.

3. The multi-layer oral active-substance film according to claim 1, characterised in that the at least one pharmaceutically active substance is contained in the at least one water-soluble adhesive layer.

4. The multi-layer oral active-substance film according to claim 1, characterised in that the at least one pharmaceutically active substance is idebenone.

5. The multi-layer oral active-substance film according to claim 1, characterised in that the at least one hydrophilic polymer in the at least two non-adhesive layers comprises polyvinyl alcohol, polyethylene oxide, polyvinylpyrrolidone or copolymers thereof or a polyvinyl caprolactam/polyvinyl acetate/polyethylene glycol copolymer or a vinylpyrrolidone/vinyl acetate copolymer or a cellulose derivative, such as hydroxypropyl cellulose or hydroxypropyl methylcellulose, starch or a starch derivative, shellac, alginic acid, galactomannan, carrageenan and other natural gums, pullulan, xanthan, pectin and other glucans, dextran, poly(meth)acrylate, polyalkylene glycol, carboxyvinyl polymer and/or copolymers thereof.

6. The multi-layer oral active-substance film according to claim 5, characterised in that the at least two non-adhesive layers each comprise a different hydrophilic polymer selected from the group consisting of polyvinyl alcohol, polyethylene oxide, polyvinylpyrrolidone or copolymers thereof, polyvinyl caprolactam/polyvinyl acetate/polyethylene glycol copolymer, vinylpyrrolidone/vinyl acetate copolymer, a cellulose derivative, such as hydroxypropyl cellulose or hydroxypropyl methylcellulose, starch or a starch derivative, shellac, alginic acid, galactomannan, carrageenan and other natural gums, pullulan, xanthan, pectin and other glucans, dextran, poly(meth)acrylates, polyalkylene glycols, carboxyvinyl polymers and/or copolymers thereof.

7. The multi-layer oral active-substance film according to claim 1, characterised in that the at least one water-soluble polymer in the at least one water-soluble adhesive layer comprises shellac, a vinylpyrrolidone/vinyl acetate copolymer, a polyvinyl caprolactam/polyvinyl acetate/polyethylene glycol copolymer, hydroxypropyl cellulose or hydroxypropyl methylcellulose and/or polyvinylpyrrolidone.

8. The multi-layer oral active-substance film according to claim 1, characterised in that the at least one plasticiser in the at least one water-soluble adhesive layer comprises glycerol, polyethylene glycol, especially polyethylene glycol 200, and/or tributyl citrate.

9. The multi-layer oral active-substance film according to claim 1, characterised in that the at least one pharmaceutically active substance in at least one of the at least two non-adhesive layers and/or in the at least one water-soluble adhesive layer is present in each case in an amount of up to 75 wt. %, in relation to the total weight of the layer in question.

10. The multi-layer oral active-substance film according to claim 1, characterised in that the weight ratio of the at least one water-soluble polymer to the at least one plasticiser in the at least one water-soluble adhesive layer is approximately 85 to 50 to approximately 15 to 50.

11. The multi-layer oral active-substance film according to claim 1, characterised in that the at least two non-adhesive layers each have an areal density of from 20 to 200 g/m².

12. The multi-layer oral active-substance film according to claim 1, characterised in that the at least one water-soluble adhesive layer has an areal density of from 20 to 200 g/m².

13. A method for producing a multi-layer oral active-substance film, comprising the following steps
(a) providing a first non-adhesive layer containing at least one hydrophilic polymer,
(b) providing a further non-adhesive layer containing at least one hydrophilic polymer,
(c) providing a water-soluble adhesive layer containing at least one water-soluble polymer and at least one plasticiser,
(d) applying the adhesive layer from step (c) to one of the non-adhesive layers from steps (a) or (b) in order to obtain a laminate, and
(e) applying the further non-adhesive layer from steps (a) or (b) to the water-soluble adhesive layer of the laminate from step (d) so that the water-soluble adhesive layer is covered from both sides by at least one non-adhesive layer,
at least one of the layers produced in steps (a), (b) and (c) additionally containing at least one pharmaceutically active substance.

14. A multi-layer oral active-substance film obtainable by the method according to claim 13.

15. A medicinal product comprised of the multi-layer oral active-substance film according to claim 1.
